(19) United States
(12) Patent Application Publication
(10) Pub. No.: US 2010/0030251 A1
(43) Pub. Date: Feb. 4, 2010

(54) DEVICES AND METHODS FOR CROSSING CHRONIC TOTAL OCCLUSIONS

(75) Inventors: Gurpreet S. Sandhu, Rochester, MN (US); Charanjit S. Rihal, Rochester, MN (US)

Correspondence Address: MUETING, RAASCH & GEBHARDT, P.A., P.O. BOX 581336, MINNEAPOLIS, MN, 55458-1336, US

(73) Assignee: Mayo Foundation for Medical Education and Research, Rochester, MN (US)

(21) Appl. No.: 12/302,142

(22) PCT Filed: May 22, 2007
(86) PCT No.: PCT/US07/12214

§ 371 Date: Jul. 1, 2009

Related U.S. Application Data
(60) Provisional application No. 60/802,981, filed on May 24, 2006.

(51) Int. Cl. A61M 29/00; A61M 25/00

(52) U.S. Cl. 606/194; 604/264

(57) ABSTRACT

Devices and methods for crossing total chronic occlusions in body lumens (e.g., blood vessels, etc.). The devices and methods may involve guidewire assisted delivery of a device designed to both traverse a chronic total occlusion (CTO) and create a passageway through the CTO for a treatment device (e.g., an angioplasty balloon, stent, etc.). Some devices of the present invention may include an expandable sleeve constructed of material that expands in response to temperature changes. In other devices, an expandable sleeve may be provided in which two or more interlocking cylindrical sections can be arranged such that the cylindrical sections have a first, smaller diameter at which the expandable sleeve can be inserted through a CTO. The expandable sleeve also has a second, larger diameter that can be achieved by moving the two or more interlocking cylindrical sections relative to each other such that the expandable sleeve moves from the first diameter to the second diameter when the expandable sleeve is located across the CTO. In other aspects, a guide catheter may include a friction-enhanced outer surface to assist in maintaining the guide catheter in a selected location during use. The friction-enhanced property may be provided using, e.g., materials with an increased coefficient of friction, adding features to the surface of an otherwise smooth guide catheter, etc.

### RELATED APPLICATION

The present application claims the benefit of U.S. Provisional Patent Application Ser. No. 60/802,981, filed on May 24, 2006 and titled DEVICES AND METHODS FOR CROSSING CHRONIC TOTAL OCCLUSIONS, which is hereby incorporated by reference in its entirety.

The present invention relates generally to devices and methods for crossing total chronic occlusions in body lumens and, more particularly, to dilation devices and methods for creating passageways through total chronic occlusions in body lumens. The present invention also relates to friction-enhanced guide catheters that may be used with the dilation devices or other medical devices.

Percutaneous transluminal coronary angioplasty (PTCA) and stenting are examples of medical procedures used to increase blood flow through the coronary arteries and can often be used as alternatives to coronary bypass surgery. In PTCA procedures, the angioplasty balloon is inflated within the stenosed vessel, at the location of an atheroma or plaque deposit, in order to shear and disrupt the wall components of the vessel to obtain an enlarged lumen. In stenting, an endoluminal prosthesis is implanted in the vessel to maintain patency following the procedure. To initiate these procedures, one must first introduce a guidewire into the lumen of the vessel to serve as a conduit for other interventional devices, such as angioplasty balloons and stent delivery systems. This guidewire must be advanced into a position past the location of the atheroma or plaque deposit.

Guidewires used in, e.g., PTCA, are preferably capable of traversing tortuous pathways within the body, including bends, loops and branches. For this reason, guidewires need to be flexible, but they should also be sufficiently stiff to serve as a guide for other devices. In addition, they are preferably torqueable to facilitate directional changes as they are guided into position. Guidewires are typically made of stainless steel, tantalum or other suitable materials, and include a variety of different designs. For example, U.S. Pat. Nos. 3,789,841, 4,545,390 and 4,619,274 disclose guidewires in which the distal segment is tapered for greater flexibility. The tapered section may be enclosed in a wire coil, typically a platinum coil, which provides increased column strength and torqueability. Another design is identified in U.S. Pat. No. 5,095,915, where the distal segment is encased in a polymer sleeve with axially spaced grooves to provide bending flexibility.

In some cases, a vessel to be opened may be totally occluded, and even a guidewire cannot be introduced past the occlusion. This condition is typically referred to as a chronic total occlusion. It may be difficult or impossible to traverse a chronic total occlusion with a guidewire and, even if possible to traverse with a guidewire, it may difficult or impossible to advance a treatment device such as an angioplasty balloon or stent delivery device over the guidewire through the chronic total occlusion.

The standard balloon angioplasty or stent placement procedure typically involves placing a guide catheter in a selected location, e.g., the ascending aorta, to engage some anatomical feature, e.g., the ostium of the selected coronary artery, with the tip of a guide catheter. A guidewire (with a diameter of, e.g., 0.014 inches) is then advanced through the guide catheter and used to cross the lesion/occlusion using, e.g., fluoroscopic guidance. The selected device can then be advanced through the guide catheter over the guidewire to reach a lesion/occlusion.

The stability and support provided by the guide catheter can become more important if the therapeutic device (e.g., balloon, stent, etc.) is difficult to advance across tortuous vessels and/or tight lesions. Pushing too hard on the therapeutic device being delivered through the guide catheter can cause the guide catheter to slip backward (proximally) causing misplacement of the guidewire and the therapeutic device, requiring the practitioner to begin again with placement of the guidewire followed by advancement of the therapeutic device.

To address that issue, practitioners typically try to maintain a stable position with the guide catheter using their left hand while they advance a therapeutic device with their right hand (or vice versa). This technique works well in many instances, but, in some cases, the practitioner has difficulty finding a stable location for the guide catheter. In those instances, the guide catheter may continue slipping too far distally or proximally (e.g., too far down the aorta or back up into the ascending aorta) as the practitioner attempts to find a stable location.

### SUMMARY

The present invention provides devices and methods for crossing total chronic occlusions in body lumens (e.g., blood vessels, etc.). The devices and methods preferably involve guidewire assisted delivery of a device designed to both traverse a chronic total occlusion (CTO) and create a passageway through the CTO for a treatment device (e.g., an angioplasty balloon, stent, etc.).

In various embodiments, the devices of the present invention may include, e.g., an expandable sleeve constructed of material that expands in response to temperature changes. Typically, such materials expand when heated. The devices may preferably include thermoelectric expandable sleeves that can be heated using electric energy (using, e.g., electrical resistance heating elements, Peltier devices, etc.). If heating is accomplished using electrical energy, it may be preferred that the electric energy be delivered using one or more electric conductor elements that extend from an electric power source to the heating element.

The thermally expandable materials that may be used in the expandable sleeves may include, e.g., metals (such as, e.g., copper, copper alloys, aluminum, gold, nickel, stainless steel, etc.), expandable gels/polymers (see, e.g., U.S. Pat. Nos. 5,403,893 to Tanaka et al., 5,961,492 to Kriesel et al., and 6,030,634 to Wu et al.). In some instances, it may be desirable to incorporate elements in the expandable sleeve in the form of bimetallic strips or other combinations of materials in which different thermal expansion coefficients may be beneficially exploited).

In other embodiments of the present invention, an expandable sleeve may be provided in which two or more interlocking cylindrical sections can be arranged such that the cylindrical sections have a first, smaller diameter at which the expandable sleeve can be inserted through a CTO. The expandable sleeve also has a second, larger diameter that can be achieved by moving the two or more interlocking cylindrical sections relative to each other such that the expandable sleeve moves from the first diameter to the second diameter when the expandable sleeve is located across the CTO.

The present invention may also include a guide catheter that includes a friction-enhanced outer surface to assist in maintaining the guide catheter in a selected location during use. The friction-enhanced outer surface may assist a practitioner by increasing resistance to movement of the guide catheter when the friction-enhanced outer surface of the guide catheter is acting against an internal body surface. That additional resistance to movement may be helpful when, e.g., advancing a dilation device of the present invention through a CTO, placing a therapeutic device (e.g., balloon, stent. etc.) across the CTO (or other lesion/occlusion), etc. The friction-enhanced property may be provided using, e.g., materials with an increased coefficient of friction, adding features to the surface of an otherwise smooth guide catheter, etc.

In one aspect, the present invention provides a chronic total occlusion expansion device that includes an elongated member having a proximal end and a distal end; a thermoelectric expandable sleeve on the elongated body proximate the distal end of the elongated body, wherein the thermoelectric expandable sleeve comprises a first diameter and a second diameter larger than the first diameter when the thermoelectric expandable sleeve is heated above a selected temperature; an electric conductor extending from the thermoelectric expandable sleeve towards the proximal end of the elongated body; and an optional electric power source adapted for electrical connection to the electric conductor element to provide electric energy to raise the temperature of the thermoelectric expandable sleeve; wherein raising the temperature of the thermoelectric expandable sleeve to the selected temperature or higher causes the thermoelectric expandable sleeve to expand from the first diameter to the second diameter.

In another aspect, the present invention provides a chronic total occlusion expansion device that includes an elongated body having a proximal end and a distal end, the proximal end and distal end defining a longitudinal axis extending along the elongated body; an expandable sleeve on the elongated body proximate the distal end of the elongated body, wherein the expandable sleeve comprises two or more interlocking sections having a first diameter when the two or more interlocking sections are in a first configuration such that and a second diameter when the two or interlocking sections are in a second configuration, wherein the second diameter is greater than the first diameter, and further wherein moving the two or more interlocking sections between the first configuration and the second configuration comprises moving the interlocking sections such that they are offset from each other along the longitudinal axis.

In another aspect, the present invention provides a chronic total occlusion expansion device that includes an elongated body having a proximal end and a distal end, the proximal end and distal end defining a longitudinal axis extending along the elongated body; a tapered screw operably attached to the elongated body; and an expandable sleeve on the elongated body proximate the distal end of the elongated body, wherein the expandable sleeve includes two or more interlocking sections having a first diameter when the two or more interlocking sections are in a first configuration such that and a second diameter when the two or more interlocking sections are in a second configuration, wherein the second diameter is greater than the first diameter, and further wherein moving the two or more interlocking sections between the first configuration and the second configuration comprises driving the tapered screw along the longitudinal axis through the two or more interlocking sections.

In another aspect, the present invention provides a guide catheter including a tubular body having a length extending between a proximal end and a distal end; and a lumen extending through the tubular body, the lumen having an opening at the distal end of the tubular body; wherein an outer surface of the tubular body proximate the distal end has a structured surface capable of enhancing friction between the tubular body and tissue in contact therewith.

In another aspect, the present invention provides a guide catheter having a tubular body with a length extending between a proximal end and a distal end; and a lumen extending through the tubular body, the lumen having an opening at the distal end of the tubular body; wherein an outer surface of the tubular body proximate the distal end includes a friction-enhanced portion that has a static coefficient of friction that is higher than the static coefficient of friction of the outer surface of the guide catheter surrounding the friction-enhanced portion.

The above summary is not intended to describe each embodiment or every implementation of the present invention. Rather, a more complete understanding of the invention will become apparent and appreciated by reference to the following Description of Exemplary Embodiments of the Invention and claims in view of the accompanying figures of the drawing.

### BRIEF DESCRIPTIONS OF THE FIGURES

FIG. 1 depicts one method of use of a dilation device according to the present invention in which a guidewire is advanced to the proximal side of a CTO and a dilation device is advanced over the guidewire towards the CTO.

FIG. 2 depicts advancement of the dilation device such that the dilation device traverses the proximal and distal ends of the CTO.

FIG. 3 depicts the expandable sleeve of the dilation device after expansion of while the expandable sleeve is located within the CTO.

FIG. 4 depicts the CTO after a passageway is formed therethrough by removal of the dilation device after its expandable sleeve has been expanded within the CTO and collapsed for removal.

FIG. 5 is a side elevational view of one example of a dilation device including a thermally expandable sleeve before expansion.

FIG. 6 is a side elevational view of the dilation device of FIG. 5 after expansion of the expandable sleeve.

FIG. 7 is a view of another example of a dilation device including interlocking cylindrical sections in the expandable sleeve (before expansion).

FIG. 8 is a view of the dilation device of FIG. 7 after expansion.

FIG. 9A depicts one exemplary actuator that may be used in connection with some of the dilation devices of the present invention.

FIG. 9B depicts another exemplary actuator that may be used in connection with some of the dilation devices of the present invention.

FIG. 10 is a side elevational view of another exemplary dilation device according to the present invention.

FIG. 11 depicts one example of a guide catheter including a friction-enhanced outer surface according to another aspect of the present invention.

FIG. 12 is an enlarged cross-sectional view of a portion of a guide catheter including structure on the friction-enhanced portion of the guide catheter.

### DESCRIPTION OF EXEMPLARY EMBODIMENTS OF THE INVENTION

In the following detailed description of exemplary embodiments of the invention, reference is made to the accompanying figures which form a part hereof, and in which are shown, by way of illustration, exemplary embodiments in which the invention may be practiced. It is to be understood that other embodiments may be utilized and structural changes may be made without departing from the scope of the present invention.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably. Thus, for example, a metal can include one or more metals, etc.

The term “and/or” means one or all of the listed elements or a combination of any two or more of the listed elements.

FIGS. 1-4 depict methods in which dilation devices according to the present invention may be used to traverse a CTO (chronic total occlusion) in accordance with the present invention. As depicted in FIG. 1, the vessel 10 may include a CTO 12 located therein that completely or nearly completely closes the vessel 10 such that fluids (e.g., blood, etc.) cannot pass through the vessel 10. In addition to presenting a barrier to the passage of fluids through the vessel, the CTO 12 may also prevent passage of a conventional guidewire through the vessel to allow for the delivery of a device that could be used to expand the CTO 12 to allow fluids to pass therethrough.

In FIG. 1, a guidewire 20 has been advanced through the vessel 10 such that the distal end 22 of the guidewire 20 is located at or near the proximal side 13 of the CTO 12. With the guidewire 20 in position at the proximal side 13 of the CTO 12, a dilation device 30 is preferably advanced over the guidewire 20 until the distal end 32 of the dilation device 30 is positioned at the proximal side 13 of the CTO 12. The guidewire 20 preferably passes through a guidewire lumen 24 in elongated body 40 that passes through the dilation device 30. The guidewire lumen 24 preferably includes an opening at the distal end 32 of the dilation device 30 such that the guidewire 20 can extend through the dilation device 30.

Although the use of a guidewire 20 is discussed in connection with the dilation devices of the invention, it should be understood that the use of a guidewire to guide the dilation device 30 to the CTO 12 is optional, i.e., in some instances, the dilation device 30 may be advanced to the CTO 12 without the assistance of a guidewire.

The dilation device 30 preferably includes an expandable sleeve 34 that extends over a portion of the longitudinal length (as measured along axis 11) of the dilation device 30. The expandable sleeve 34 may take a variety of forms, some examples of which are described herein. The distal end 32 of the dilation device 30 may preferably be tapered or otherwise shaped to facilitate advancement of the dilation device 30 through the CTO 12. The dilation device 30 may also optionally include one or more cutting elements 36 located on its distal end 32 to assist in advancement of the dilation device 30 through the CTO 12.

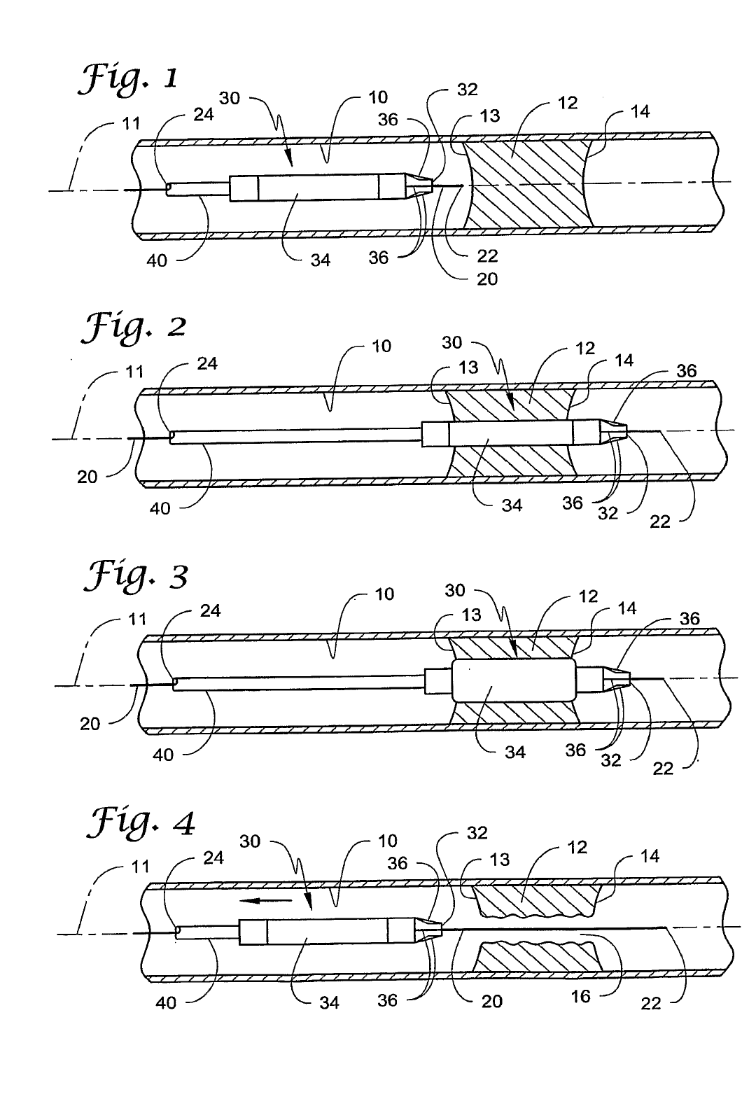

With the dilation device 30 in position at the proximal side 13 of the CTO 12, the distal end 32 of the dilation device 30 is preferably advanced into the CTO 12 until the expandable sleeve 34 is embedded in the CTO 12. It may be preferred that the expandable sleeve 34 of the dilation device 30 be long enough to traverse the CTO 12 such that the distal end of the expandable sleeve 34 extends past the distal side 14 of the CTO 12 while the proximal end of the expandable sleeve 34 remains outside the proximal side 13 of the CTO 12 as depicted in FIG. 2. The optional blade or blades 36 on the distal end 32 of the dilation device 30 may preferably assist in advancement of the dilation device 30 through the CTO 12.

With the dilation device in position as seen in FIG. 2, the expandable sleeve 34 of the dilation device 30 is preferably expanded from its smaller first diameter to a larger second diameter as depicted in FIG. 3. Expansion of the expandable sleeve 34 preferably further opens the passageway 16 formed by advancing the dilation device 30 through the CTO 12.

It may be preferred that the larger second diameter of the expandable sleeve 34 be, e.g., 105% percent or more (in some instances 110% or more) of the smaller first diameter of the expandable sleeve 34 to facilitate passage of a guidewire 20 through the passageway 16 traversing the CTO 12 after expansion by the dilation device 30. At the upper end, it may be preferred that the expandable sleeves 34 used in connection with dilation devices 30 of the present invention have larger second diameters that are, e.g., 200% or less (in some instances, 150% or less) of the smaller first diameter of the expandable sleeve 34.

It may also be preferred that expansion of the expandable sleeve 34 of the dilation device 30 be reversible. In other words, it may be preferred that the expandable sleeve 34 be collapsible back to at least near the smaller first diameter to assist in removal of the dilation device 30 from the CTO 12 (and the vessel 10 into which it was inserted to reach the CTO 12). As depicted in FIG. 4, the expandable sleeve 34 has been collapsed and withdrawn from the CTO 12, leaving passageway 16 through the CTO 12. It may further be preferred that the guidewire 20 be advanced through the passageway 16 in the CTO 12 before the dilation device 30 is withdrawn from the CTO 12. By doing so, the guidewire lumen of the dilation device 30 can be used to guide the guidewire 20 through the passageway 16 such that the distal end 22 of the guidewire 20 is located past the distal side 14 of the CTO 12.

The expandable sleeves used in dilation devices of the present invention may take on a variety of forms. Typically, however, the dilation devices preferably have (unexpanded) diameters that are smaller than those found in (unexpanded) devices designed to expand CTO's to therapeutically acceptable levels. In other words, the advantage of using a dilation device according to the present invention before a conventional dilation/expansion device (such as, e.g., a balloon) is the smaller diameter of the dilation devices of the present invention.

Another potential advantage of using a dilation device according to the present invention is that the dilation device may preferably be more rigid or stiff than a conventional dilation/expansion device (e.g., a balloon). That increased rigidity/stiffness, when combined with the optional cutting blades 36 may enhance the device's ability to cross a CTO (as compared to a conventional dilation/expansion device).

The expandable sleeves used in dilation devices of the present invention may be operated using a number of different expansion technologies. It may, however, be preferred that the dilation devices do not rely on fluidic expansion (such as that used in, e.g., balloons) to limit the need to provide fluid delivery lumens along the length of the dilation device. Such lumens typically increase the profile size of the dilation device. As used in connection with the present invention, the phrase “non-fluidic expandable sleeve” means an expandable sleeve that can be expanded without the use of fluid (e.g., gas, liquid, etc.).

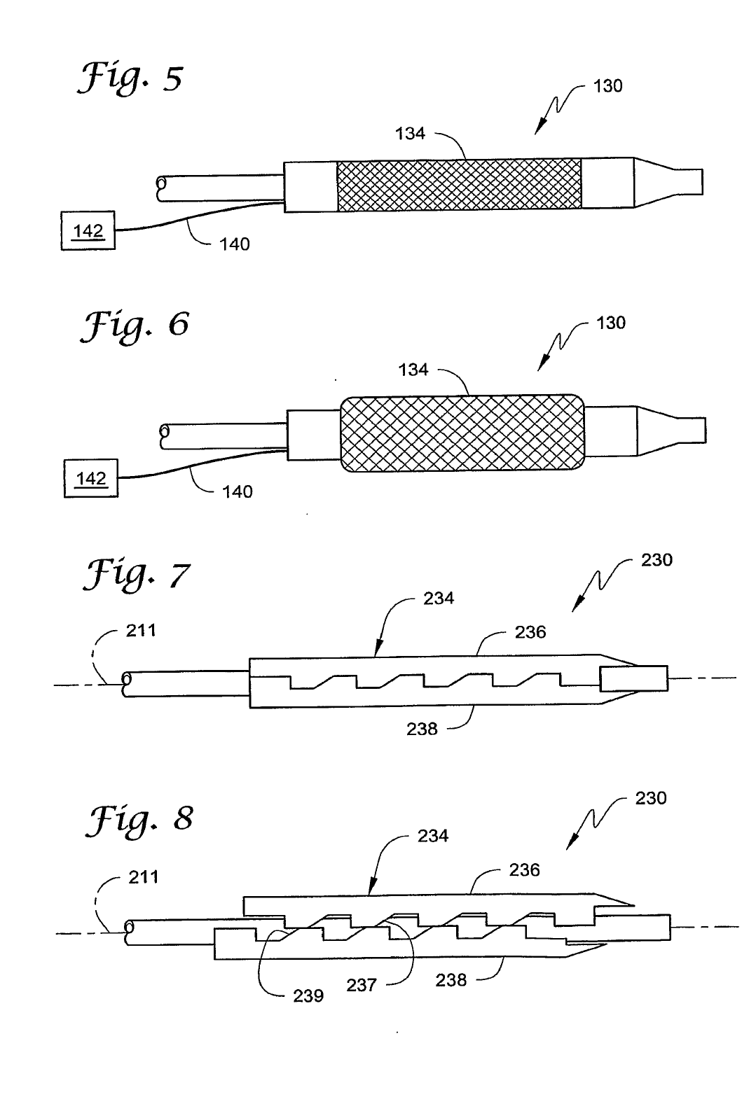

FIG. 5 depicts on example of an expandable sleeve 134 in the form of a thermally expandable sleeve comprising one or more thermally expandable elements wound about the dilation device 130 in a manner that allows for expansion of the expandable sleeve from a smaller first diameter (seen in FIG. 5) to a larger second diameter (depicted in FIG. 6) when the expandable sleeve 130 is heated above a selected temperature. The windings may be, e.g., in a crossing helical wound pattern, concentric helical pattern, etc. The thermally expandable elements may preferably form a thermally expandable sleeve in the form of a an expandable mesh.

It may be preferred that the thermal energy used to heat the thermally expandable sleeve be provided using electric resistance heating. In such a system, the dilation device 130 may preferably include at least one electrical conductor 140 extending from the proximal end of the expandable sleeve towards the proximal end of the dilation device 130 where it can be connected to an electric power source 142 to provide electric energy to raise the temperature of thermally expandable sleeve 134 to the selected temperature.

As discussed herein, raising the temperature of the thermally expandable sleeve to the selected temperature (or higher) preferably causes the expandable sleeve to expand from its smaller first diameter (FIG. 5) to a larger second diameter (FIG. 6). The expansion is preferably the result of volumetric thermal expansion experienced by most materials when heated. Thermal expansion properties of many materials are well-defined and typically can be quantified using the volumetric thermal expansion coefficient (sometimes referred to as the thermal expansion coefficient or coefficient of thermal expansion) and the principles behind that thermodynamic phenomenon will not be discussed herein.

It may be preferred that the expandable sleeve 134 be constructed of metals or other electrically conductive materials that can serve as both the expandable sleeve elements and that also serve as thermal heating elements by virtue of their inherent resistance to the passing of an electric current. Expandable sleeves 134 constructed of elements that serve the dual functions of both heating elements and structural expansion elements may be referred to herein as thermoelectric expandable sleeves.

It may be preferred that the materials used in thermoelectric expandable sleeves of the present invention exhibit a relatively high coefficient of thermal expansion. For example, it may be preferred that the materials used in a thermoelectric expandable sleeve used in a dilation device of the present invention have a thermal expansion coefficient of about 10 or more. Examples of some potentially suitable materials that may be used include, e.g., platinum, copper, aluminum, gold, nickel, stainless steel, etc. Alloys of at least some of these different materials may also be acceptable (e.g., copper alloys, etc.). In some embodiments, it may be preferred that the thermoelectric expandable sleeve consist essentially of one or metals with a coefficient of thermal expansion of about 10 or more.

The elements used to manufacture the expandable sleeve 134 may, in some embodiments, be provided as bimetallic strips in which two materials (e.g., metals, metallic alloys, etc.) are provided in such a manner as to take advantage of differences in the thermal expansion coefficients of the different materials.

Such bimetallic strips may be used to enhance the formation of curved surfaces in the expandable sleeve during expansion.

Although not depicted in FIGS. 5 & 6, it may be preferred to provide a non-electrically conductive protective cover over the thermoelectric expandable sleeve to reduce unwanted conduction of electrical energy into the tissue surrounding the sleeve 134 during the heating process.

In another exemplary embodiment, the expandable sleeve 134 may be constructed of a gel/polymer material that exhibits thermal expansion properties as opposed to the thermoelectric expandable sleeves described herein. Preferably, the thermal expansion results from heating the gel/polymer to a selected temperature. Examples of such materials may be found in, e.g., U.S. Pat. Nos. 4,732,930 (Tanaka et al.); Re 35,068 (Tanaka et al.); 5,403,893 (Tanaka et al.); 5,961,492 (Kriesel et al.); and 6,030,634 (Wu et al.).

As with the thermoelectric expandable sleeves described herein, the heating may preferably be accomplished through the use of electrical resistance heating element. If the gel/polymer does not, itself, function as the heating element, it may be preferred to incorporate one or more electrical resistance heating elements in the dilation device 130 such that the temperature of the gel/polymer in the expandable sleeve 134 can be raised to a selected temperature at which the selected amount of expansion can be achieved.

Regardless of the materials used to construct the expandable sleeve 134, it may be preferred that expansion of the expandable sleeve 134 be reversible. In other words, it may be preferred that the thermal expansion reverse when cooled such that the expandable sleeve 134 collapses to a size that is the same as or nearly the same as its smaller first diameter. It may be preferred, for example, that the expandable sleeve not exceed any elastic yield limits during the expansion.

FIGS. 7 & 8 depict another exemplary embodiment of a dilation device 230 that may be used in connection with the present invention. The dilation device 230 preferably expands as result of mechanical interference between two or more sections of an expandable sleeve 234. It may be preferred that the sections 236 and 238 be formed as interlocking sections such that offsetting the sections either longitudinally (along longitudinal axis 211) or rotationally (around longitudinal axis 211) results in radial expansion of the expandable sleeve from a smaller first diameter to a larger second diameter.

Regardless of the motion required to expand the sleeve 234, it may be preferred that the dilation device 230 include an actuating member operably attached to one or more of the sections of the sleeve 234. The actuator preferably extends towards the proximal end of the dilation device 230, whereby the actuating member is capable of moving the interlocking sections relative to each other (longitudinally or rotationally).

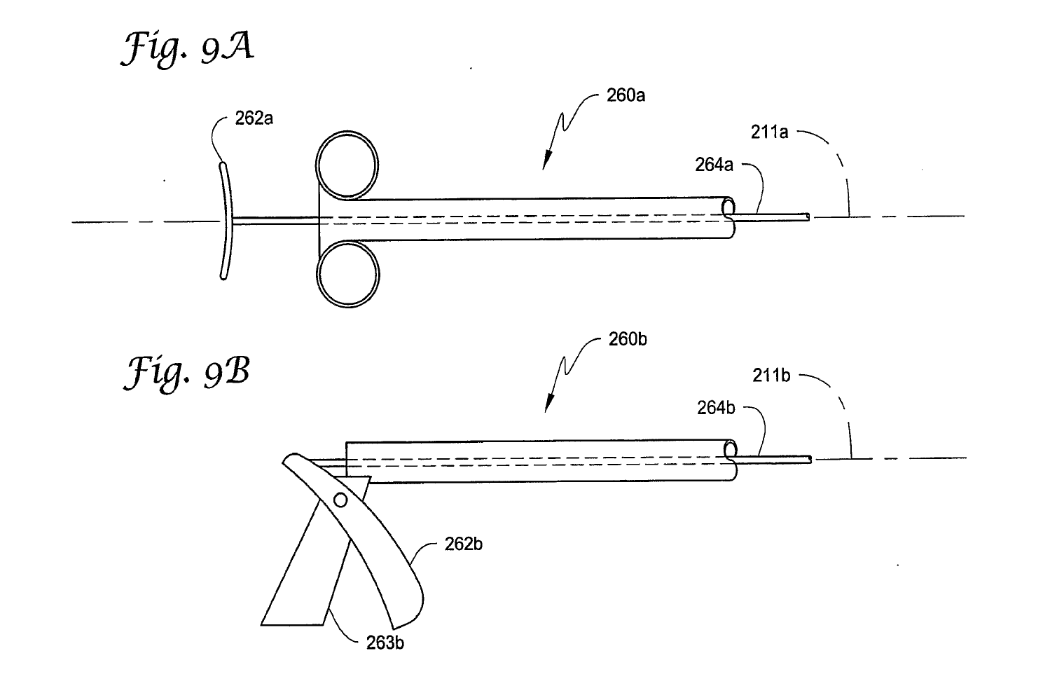

Examples of some potentially suitable actuators are depicted in FIGS. 9A & 9B, although many other actuators could be used in place of those depicted. The actuator 260a in FIG. 9A includes a working end 262a that transfers motion via element 264a that preferably extends distally along longitudinal axis 211a to the dilation device (not shown in FIG. 9A). The working end 262a may be used as a push/pull assembly to transfer translational motion to the dilation device along longitudinal axis 211a using element 264a to transfer that force. Alternatively, the working end 262a may be used to rotate element 264a about longitudinal axis 211a, with that rotation being transferred to the dilation device operably attached to element 264a. In some instances, both translational and rotational motion may be transferred using element 264a.

The alternate exemplary actuator 260b depicted in FIG. 9B includes handles 262b & 263b hingedly connected to each other and operably attached to element 264b to transfer motion along the longitudinal axis 211b to a dilation device (not shown in FIG. 9B). The motion transferred along element 264b may be translational (e.g., pushing, pulling), rotational, or both with the use of appropriate mechanical structures (e.g., gears, racks, pinions, etc.).

Any actuator mechanism used in connection with the dilation devices of the present invention may preferably be designed to facilitate the use of a guidewire in connection with the dilation device. As for the actuators depicted in FIGS. 9A & 9B, a guidewire lumen could potentially be incorporated into the element 264a/264b. Other techniques to accommodate guidewires could also be used.

Returning to FIGS. 7 & 8, in the depicted embodiment, the sections 236 and 238 interlock in a manner that includes cooperating ramps 237 & 239 (respectively) to assist in movement of the sections 236 and 238 longitudinally relative to each other. That longitudinal offset causes the outer surfaces of the sections 236 & 238 to move radially outward from the longitudinal axis 211, with the cooperating ramps 237 & 239 sliding over each other.

The geometry of the interlocking sections 236 & 238 may be designed to result in a selected amount of radial expansion in the sleeve 234. It may, for example, be preferred that the larger second diameter be about 50% larger than the smaller (unexpanded) first diameter. As with the expandable sleeves discussed above, it may be preferred that the expansion be reversible, i.e., that the sections 236 & 238 be movable to collapse the expandable sleeve 234.

The sections 236 & 238 of the expandable sleeve 234 may be constructed of a variety of different materials. Some examples of potentially suitable materials may include, e.g., metals (such as titanium, etc.), polymers, composites, etc.

Although the expandable sleeve 234 is depicted as constructed of two sections 236 & 238, it should be understood that the mechanically expandable sleeves including interlocking sections may be constructed in three or more sections if so desired.

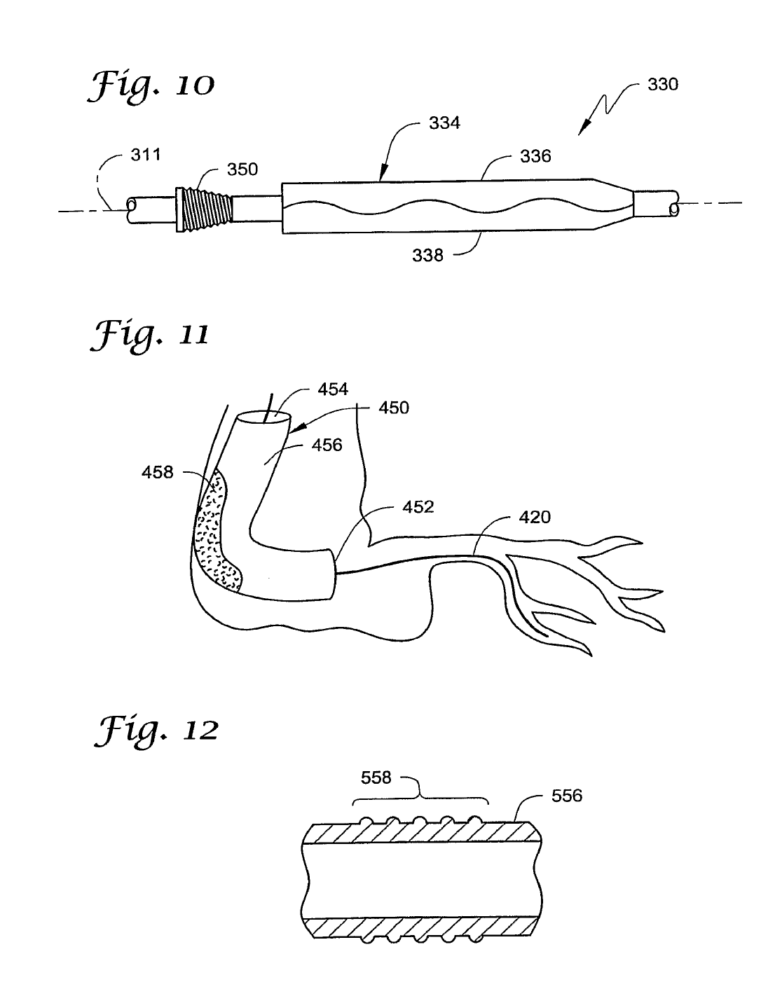

Referring to FIG. 10, in place of ramps and longitudinal movement of interlocking sections, mechanical expansion of an expandable sleeve may alternatively be accomplished by advancing a tapered screw 350 through an expandable sleeve 334 that includes two (or more) sections 336 & 338 in a dilation device 330. As the screw 350 is advanced through the sections 336 & 338, the sections expand radially outward from the longitudinal axis 311.

The dilation device 330 may preferably include an actuating member operably attached to the tapered screw 350 and extending towards the proximal end of the dilation device 330, whereby the actuating member is capable of rotating the tapered screw about the longitudinal axis.

The geometry of the screw 350 and the interlocking sections 336 & 338 may be designed to result in a selected amount of radial expansion in the sleeve 334. It may, for example, be preferred that the larger second diameter be about 50% larger than the smaller (unexpanded) first diameter. As with the expandable sleeves discussed above, it may be preferred that the expansion be reversible, i.e., that the sections 336 & 338 and the screw 350 be movable to collapse the expandable sleeve 334.

Although FIG. 10 depicts the use of a threaded screw 350 to force the interlocking sections 336 & 338 apart, it should be understood that similar actions may be accomplished using, e.g., a tapered wedge (without threads) that is forced in between the sections 336 & 338 to force the sections apart.

The screw 350 and sections 336 & 338 of the expandable sleeve 334 may be constructed of a variety of different materials. Some examples of potentially suitable materials may include, e.g., metals (such as titanium, etc.), polymers, composites, etc.

Although the expandable sleeve 334 is depicted as constructed of two sections 336 & 338, it should be understood that the mechanically expandable sleeves including interlocking sections may be constructed in three or more sections if so desired.

In another variation, although the dilation devices of the present invention may preferably include a guidewire lumen or other structure to accommodate the use of a separate guidewire in conjunction with the dilation device, other variations are possible. For example, the expandable sleeve and any required structure may be integrated directly within the guidewire structure itself (preferably proximate the distal end of the guidewire). In such an embodiment, the guidewire could be advanced and dilation performed using the guidewire and integrated expandable sleeve.

The integrated expandable sleeve and associated components may preferably be removable from the guidewire in situ to allow passage of treatment devices over the guidewire (where those devices could not otherwise pass over the guidewire to reach the CTO with the expandable sleeve and its components in place). Alternatively, only the components proximal to the expandable sleeve may need to be removed to allow additional devices to be passed over the guidewire to reach the lesion. In another alternative, the guidewire with its integrated expandable sleeve could be advanced distally past the CTO after dilation such that a treatment device could be advanced into the CTO without encountering the integrated expandable sleeve on the guidewire (which would be located distal of the CTO).

FIGS. 11-12 depict various exemplary embodiments of guide catheters that may be used in connection with the present invention. FIG. 11 depicts a guide catheter 450 deployed within the aorta of a subject and a guidewire 420 deployed through the guide catheter 450 into a coronary artery. Although the depicted guide catheter 450 is deployed within the aorta, it should be understood that the guide catheters of the present invention may be used in any selected location within the body where the friction-enhanced properties of the guide catheter 450 may be beneficial.

The guide catheter 450 includes a elongated body extending from a proximal end to a distal end 452. The elongated body may preferably include at least one lumen 454 extending to the distal end 452, where the lumen 454 includes an opening through which various devices can be delivered. The guide catheter 450 has an outer surface 456 that includes a friction-enhanced portion 458. It may be preferred that the friction-enhanced portion 458 of the outer surface of the guide catheter 450 be located proximate the distal end 452 of the guide catheter 450.

When deployed within, e.g., the aorta as seen in FIG. 11, it may be preferred that the friction-enhanced portion 458 be positioned to contact the back wall of the aorta.

In some respects, the friction-enhanced portion 458 of the guide catheter 450 may be most simply described as having a static coefficient of friction that is higher (preferably two times or more as high) as the static coefficient of friction of the surrounding outer surface of the guide catheter 450 (where the surrounding outer surface is that portion of the outer surface surrounding the friction-enhanced portion 458). The following discussion provides some examples as to how to achieve the increased static coefficient of friction (e.g., using different materials, physical structures, etc.).

Although the friction-enhanced portion 458 of the outer surface of the guide catheter 450 is depicted as extending only partially around the circumference of the guide catheter 450, it should be understood that the friction-enhanced portion of the outer surface could alternatively extend completely around the circumference of the guide catheter 450.

The friction-enhanced portion 458 of the guide catheter 450 may be provided by a variety of techniques. Some potentially suitable techniques may include, e.g., non-slip materials (rubbers, etc.), structured surfaces, particles embedded in the outer surface of the guide catheter, etc. In some instances, combinations of two or more of these techniques may be used together.

If a non-slip material is used to provide the friction-enhanced portion 458, the selected material may be, e.g., insert molded into the catheter 450, applied to the outer surface of the catheter 450 after the body of the catheter is formed, etc. It may be preferred that any non-slip materials used be atraumatic. Because the anti-slip material is used for its friction-enhancing properties, it need only be as thick as required to achieve that result, e.g., in many instances, the non-slip material may be only a few micrometers thick.

FIG. 12 is an enlarged cross-sectional view of one potential structured surface that may be used for the friction-enhanced portion of a guide catheter in accordance with the present invention. The structured surface 558 may include, e.g., ribs molded into the outer surface 556 of the guide catheter. Although ribs are depicted in FIG. 12 may be annular, other rib orientations and shapes may be used.

Furthermore, annular ribs are only one option among many for providing a structure in the outer surface of the guide catheters of the invention. The structures used may include, e.g., rounded bumps, pyramids, posts, grooves, channels, etc. In addition to deforming the outer surface of the catheter (from an otherwise smooth surface found in conventional catheters), the structure in a friction-enhanced portion may be provided by embedding particles in the outer surface of the catheter (or adhesively attaching particles to the outer surface of the catheter). Such particles may be exposed on the outer surface of the guide catheter or they may be completely encased within the material of the outer surface of the guide catheter. Other options include, e.g., increasing roughness of the outer surface by, e.g., mechanical abrasion, chemical etching, solvent etching, etc.

Although the guide catheters discussed herein are described as including only one friction-enhanced portion, it should be understood that guide catheters of the present invention may include more than one discrete area in which the friction of the outer surface of the guide catheter is enhanced. In another alternative, although it may be preferred that only a portion (or portions) of the outer surface of the guide catheter is friction-enhanced, in some embodiments all of the outer surface of the working length of the guide catheter (where the working length is that portion of the catheter designed for insertion into a body during use) may be friction-enhanced as discussed herein. The friction-enhanced portions may be limited in circumferentially (i.e., the friction-enhanced portions may extend only partially around the circumference of a guide catheter) and/or the friction-enhanced portions may be limited longitudinally (i.e., the friction-enhanced portions may extend along only a portion of the longitudinal length of the guide catheter).

The complete disclosure of the patents, patent documents, and publications cited herein are incorporated by reference in their entirety as if each were individually incorporated.

Exemplary embodiments of this invention are discussed and reference has been made to some possible variations within the scope of this invention. These and other variations and modifications in the invention will be apparent to those skilled in the art without departing from the scope of the invention, and it should be understood that this invention is not limited to the exemplary embodiments set forth herein. Accordingly, the invention is to be limited only by the claims provided below and equivalents thereof.

1. A chronic total occlusion expansion device comprising:
an elongated body comprising a proximal end and a distal end;
a thermoelectric expandable sleeve on the elongated body proximate the distal end of the elongated body, wherein the thermoelectric expandable sleeve comprises a first diameter and a second diameter larger than the first diameter when the thermoelectric expandable sleeve is heated above a selected temperature;
an electric conductor extending from the thermoelectric expandable sleeve towards the proximal end of the elongated body; and
an optional electric power source adapted for electrical connection to the electric conductor element to provide electric energy to raise the temperature of the thermoelectric expandable sleeve;
wherein raising the temperature of the thermoelectric expandable sleeve to the selected temperature or higher causes the thermoelectric expandable sleeve to expand from the first diameter to the second diameter.

2. A device according to claim 1, wherein the thermoelectric expandable sleeve comprises a metal, and wherein, optionally, the metal has a coefficient of thermal expansion of about 10 or more.

3. A device according to claim 1, wherein the thermoelectric expandable sleeve consists essentially of a metal with a coefficient of thermal expansion of about 10 or more.

4. A device according to claim 1, wherein the thermoelectric expandable sleeve comprises a bimetallic element.

5. A device according to claim 1, wherein the thermoelectric expandable sleeve comprises an expandable gel/polymer.

6. A device according to claim 1, wherein the expandable sleeve comprises a mesh.

7. A device according to claim 1, wherein the second diameter of the thermoelectric expandable sleeve is 105% or more of the first diameter of the thermoelectric expandable sleeve.

8. A device according to claim 1, further comprising one or more cutting blades proximate the distal end of the elongated body.

9. A chronic total occlusion expansion device comprising:
an elongated body comprising a proximal end and a distal end, the proximal end and distal end defining a longitudinal axis extending along the elongated body;
an expandable sleeve on the elongated body proximate the distal end of the elongated body, wherein the expandable sleeve comprises two or more interlocking sections having a first diameter when the two or more interlocking sections are in a first configuration such that and a second diameter when the two or interlocking sections are in a second configuration, wherein the second diameter is greater than the first diameter, and further wherein moving the two or more interlocking sections between the first configuration and the second configuration comprises moving the interlocking sections such that they are offset from each other along the longitudinal axis.

10. A device according to claim 9, wherein the expandable sleeve comprises only two interlocking sections.

11. A device according to claim 9, wherein each interlocking section of the two or more interlocking sections comprises a plurality of ramp surfaces.

12. A device according to claim 9, wherein one interlocking section of the two or more interlocking sections comprises an actuating member operably attached thereto and extending towards the proximal end of the elongated body, whereby the actuating member is capable of translating the one interlocking section in a direction aligned with the longitudinal axis.

13. A device according to claim 9, wherein the two or more interlocking sections comprise one or more cutting blades proximate the distal end of the elongated body.

14. A chronic total occlusion expansion device comprising:
an elongated body comprising a proximal end and a distal end, the proximal end and distal end defining a longitudinal axis extending along the elongated body;
a tapered screw operably attached to the elongated body; and
an expandable sleeve on the elongated body proximate the distal end of the elongated body, wherein the expandable sleeve comprises two or more interlocking sections having a first diameter when the two or more interlocking sections are in a first configuration such that and a second diameter when the two or more interlocking sections are in a second configuration, wherein the second diameter is greater than the first diameter, and further wherein moving the two or more interlocking sections between the first configuration and the second configuration comprises driving the tapered screw along the longitudinal axis through the two or more interlocking sections.

15. A device according to claim 14, wherein the expandable sleeve comprises only two interlocking sections.

16. A device according to claim 14, further comprising an actuating member operably attached thereto and extending towards the proximal end of the elongated body, whereby the actuating member is capable of rotating the tapered screw about the longitudinal axis.

17. A device according to claim 14, wherein the two or more interlocking sections comprise one or more cutting blades proximate the distal end of the elongated body.

18-24. (canceled)

25. A guide catheter comprising:
a tubular body comprising a length extending between a proximal end and a distal end;
a lumen extending through the tubular body, the lumen comprising an opening at the distal end of the tubular body;
wherein an outer surface of the tubular body proximate the distal end includes a function-enhanced portion that has a static coefficient of friction that is higher than the static coefficient of friction of the outer surface of the guide catheter surrounding the friction-enhanced portion.

26. A guide catheter according to claim 25, wherein the friction-enhanced portion comprises a structured surface.

27. A guide catheter according to claim 26, wherein the structured surface comprises ribs formed in the outer surface.

28. A guide catheter according to claim 25, wherein the structured surface comprises particles embedded in the outer surface of the tubular body.

29. A guide catheter according to claim 25, wherein the friction-enhanced portion extends along only a portion of the length of the tubular body.

30. A guide catheter according to claim 25, wherein the friction-enhanced portion extends about only a portion of the circumference of the tubular body.
